(19) United States
(12) Patent Application Publication
(10) Pub. No.: US 2013/0131551 A1
(43) Pub. Date: May 23, 2013

(54) METHODS AND DEVICES FOR DIAGNOSING AND TREATING VOCAL CORD DYSFUNCTION

(71) Applicants: Shriram Raghunathan, Houston, TX (US); Marc Kelly, Indianapolis, IN (US); Jessica Hagen, Lafayette, IN (US); Omar Abdel-Latief, Lafayette, IN (US)

(72) Inventors: Shriram Raghunathan, Houston, TX (US); Marc Kelly, Indianapolis, IN (US); Jessica Hagen, Lafayette, IN (US); Omar Abdel-Latief, Lafayette, IN (US)

(21) Appl. No.: 13/636,919

(22) PCT Filed: Mar. 24, 2011
(86) PCT No.: PCT/US11/29765

§ 371 Date: Feb. 1, 2013

Related U.S. Application Data
(60) Provisional application No. 61/316,867, filed on Mar. 24, 2010.

(51) Int. Cl. A61N 1/36; A61B 5/11

(52) U.S. Cl. CPC A61N 1/36014; A61B 5/1107
USPC 600/587; 607/62

(57) ABSTRACT

Methods for diagnosing and treating vocal cord dysfunction by recording the position of the vocal cords of a subject during a monitoring period are disclosed. Portable devices are disclosed using electronic signals to provide diagnostic data indicating a subject's closed or open vocal cords to diagnose and treat vocal cord dysfunction. Corresponding software and circuitry are also disclosed.

### CROSS REFERENCE

This application claims priority from U.S. Provisional Patent Application 61/316,867, entitled “A method and device to diagnose and treat vocal cord dysfunction,” filed on Mar. 24, 2010, the disclosure of which is expressly incorporated by reference.

### TECHNICAL FIELD

The field of this disclosure relates to methods of diagnosing and treating vocal cord dysfunction and the use of devices to diagnose and treat vocal cord dysfunction.

### BACKGROUND ART

Vocal Cord Dysfunction (“VCD”), also known as paradoxical vocal fold motion (“PVFM”), affects more than 1.3 million Americans with 40,000 new cases each year. Despite its small population, Vocal Cord Dysfunction has a significant economic impact on the health care system due to the rate and length of misdiagnosis as well as number of ongoing medical check-ups and ER visits for confirmed cases.

Vocal Cord Dysfunction occurs when the vocal cords abduct (“close”) during inspiration. When the vocal cords close, the airways are blocked so inspiration can only occur through a diamond-shaped “posterior chink.” A top view of the vocal cords of a subject during normal inspiration can be seen in FIG. 5A. Conversely the closed vocal cords and posterior chink can be seen in FIG. 5B. FIG. 5B illustrates what occurs during a vocal cord dysfunction respiratory attack.

Closure of the vocal cords blocks the airway, prevents subjects from breathing normally, and results in wheezing, shortness of breath, and in extreme cases, fainting. On average, “respiratory attack” from this dysfunction will last anywhere from 15-40 minutes depending on its severity. The dysfunction is not isolated to one particular demographic group and is often misdiagnosed as asthma due to the similarities between symptoms, especially since the environmental stimulants that can lead to respiratory attack are similar. These symptoms are shared by a number of laryngeal disorders including asthma and VCD, making diagnosis extremely difficult. Symptoms of the dysfunction are not present when the patient is not experiencing respiratory attack, making positive diagnosis of VCD very difficult when the patient is not experiencing respiratory attack.

The exact causes of VCD are unclear but it is known that there are a range of triggers that cause respiratory attacks. These triggers include stress, smoke, exercise, perfume and a number of other irritants which initiate respiratory attacks. The clinical hypothesis for the cause of respiratory attack is that mediation of the vagus nerve may alter the laryngeal tone and lower the threshold for stimuli to produce vocal cord spasm or to precipitate the normal adduction of vocal cords. (Goldman J, Muers M, “Vocal cord dysfunction and wheezing.” Thorax, June 1991;46(6):401-4). Estimates by clinicians (“investigators”) show that anywhere between 15%-40% of asthmatics that do not respond to aggressive medication therapy may actually be VCD patients. (National Jewish Center for Immunology and Respiratory Medicine (Records: 1984-91).

Medical professionals (sometimes referred to as doctors, physicians, clinicians, and/or investigators) currently have two options for assessing whether a subject has vocal cord dysfunction. The most definitive test is to trigger respiratory attack within a clinical environment and then use either a flexible fiberoptic rhinolaryngoscope to directly observe vocal cord movement or a spirometry procedure which charts the volume of air inhaled and exhaled during normal breathing. These approaches are problematic as patients experiencing VCD have difficulty pinpointing the source of constriction and triggering respiratory attack within a clinical setting requires the office to simulate a wide range of conditions necessary to spur respiratory attack. The difficulty to trigger an in-clinic respiratory attack combined with the discomfort of the procedure and the risk of misdiagnosis due to the natural gag reflex that adducts vocal cords of patients during probe insertion, results in a general reluctance or inability of clinicians to make a positive prognosis.

To compensate for this difficulty, clinicians typically deprioritize VCD testing until late in the asthma assessment period, as illustrated in FIG. 1. Lack of an effective process to diagnosis VCD leads to a four-year average period of misdiagnosis where patients cycle through two—three asthma medications and scores of medical visits.

### DISCLOSURE

The present disclosure includes a device supported by the subject or by an investigator, the device using electronic signals to provide diagnostic data indicating an area of opening between vocal folds of vocal cords of subject, whether vocal folds of vocal cords are opened or closed as an indication of vocal cord dysfunction, the device comprising a power source, a signal generator powered by the power source, the signal generator configured to generate electronic signals, a microcontroller configured to transmit, direct, or control the passage of electronic signals, at least one pair of electrodes, the at least one pair of electrodes configured to be disposed adjacent to the vocal cords of the subject, the at least one pair of vocal cords including a first electrode configured to be disposed to one side of the vocal cords and a second electrode configured to be disposed to the other side of the vocal cords, wherein the transmission of electronic signals between the at least one pair of electrodes and through the vocal cords of the subject provides diagnostic data indicating an open position or a closed position of the subject's vocal folds, and memory configured to record diagnostic data.

The present disclosure also includes a method of diagnosing and treating vocal cord dysfunction by recording the position of the vocal cords of a subject during a monitoring period, the method comprises the steps of assessing a subject for airflow obstruction during inspiration, providing the subject with a recording device which measures the area of opening between vocal folds of vocal cord, the device including at least one pair of electrodes configured to be disposed on opposite sides of the vocal cords of the subject, monitoring the subject for a period of time, transmitting electronic signals between the at least one pair of electrodes, recording data indicating an open position or a closed position of the subject's vocal cords during the monitoring period of time, and analyzing data to diagnose vocal cord dysfunction.

The present disclosure also includes a device using electronic signals to provide diagnostic data indicating a subject's closed or open vocal cords during vocal cord dysfunction, the device comprising a signal generator configured to generate a sinusoidal AC voltage waveform, a voltage-to-current converter generates a constant current amplitude and alternating voltage, a switch configured to couple the constant current amplitude and alternating voltage to an electrode, a pair of electrodes configured to transmit the constant current amplitude and alternating voltage across the vocal cords of the subject, a root-mean-square detector to measure the relative impedance across the vocal cords, a microcontroller configured to transmit, direct or digitize the measured relative impedance, and memory configured to store electronic signals.

### BRIEF DESCRIPTION OF DRAWINGS

The above-mentioned and other features of this disclosure, and the manner of attaining them, will become more apparent and the disclosure itself will be better understood by reference to the following description of embodiments of the disclosure taken in conjunction with the accompanying drawings, wherein:

FIG. 1 depicts a diagnostic and treatment flow diagram of traditional asthma assessment.

FIG. 2 depicts a diagnostic and treatment flow diagram of the assessment of FIG. 1 with earlier diagnosis and treatment of vocal cord dysfunction according to one embodiment of the present disclosure.

FIG. 3A depicts a schematic perspective view of a subject wearing a device according to one embodiment of the present disclosure.

FIG. 3B depicts a schematic diagram of the circuitry of the device of FIG. 3A according to one embodiment of the present disclosure.

FIG. 4A depicts a schematic perspective view of the open vocal cords of the subject of FIG. 3 and the circuitry of FIG. 3B according to one embodiment of the present disclosure.

FIG. 4B depicts a schematic perspective view of the closed vocal cords of the subject of FIG. 3 and the circuitry of FIG. 3B according to one embodiment of the present disclosure.

FIG. 5A depicts a schematic perspective view of a subject wearing a device according to another embodiment of the present disclosure.

FIG. 5B depicts a schematic diagram of the circuitry of the device of FIG. 4A according to another embodiment of the present disclosure.

FIG. 6A depicts a schematic perspective view of a subject interacting with a device according to yet another embodiment of the present disclosure.

FIG. 6B depicts a schematic perspective view of a detachable electrode configuration for use with any device according to any embodiment of the present disclosure.

FIG. 6C depicts an exploded view of a connector of the electrode configuration of FIG. 6B.

FIG. 7 depicts a schematic perspective view of an array of electrodes for use with any device according to any embodiment of the present disclosure.

FIG. 8 depicts a schematic flow diagram according to an alternative embodiment of the present disclosure.

FIG. 9 depicts a schematic diagram of alternative circuitry as an embodiment of the flow diagram of FIG. 8. The alternative circuitry is for use with any device according to any embodiment of the present disclosure.

FIG. 10 depicts a schematic flow diagram of the software of any device according to any embodiment of the present disclosure.

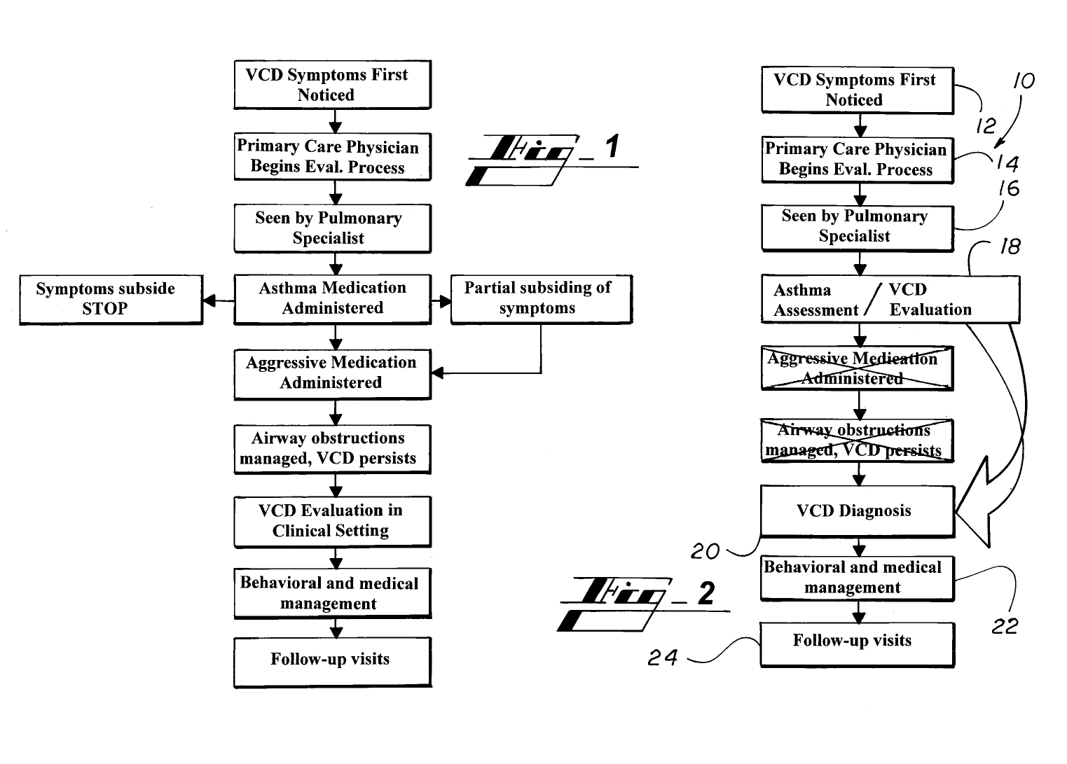

Corresponding reference characters indicate corresponding parts throughout the several views. Although the drawings represent embodiments of the present disclosure, the drawings are not necessarily to scale and certain features may be exaggerated in order to better illustrate and explain the present disclosure.

### DETAILED DESCRIPTION OF THE EXEMPLARY EMBODIMENTS

The embodiments disclosed below are not intended to be exhaustive or limit the disclosure to the precise forms disclosed in the following detailed description. Rather, the embodiments are chosen and described so that others skilled in the art may utilize their teachings.

An opportunity exists to develop a simple, relatively inexpensive diagnostic that can be used at the first step of asthma assessment to filter out those with VCD and not asthma. Due to the high time, talent, and resource costs of VCD misdiagnosis and lack of effective testing, a diagnostic process that included VCD diagnosis in connection with the initial round of asthma assessment may greatly improve patient outcomes.

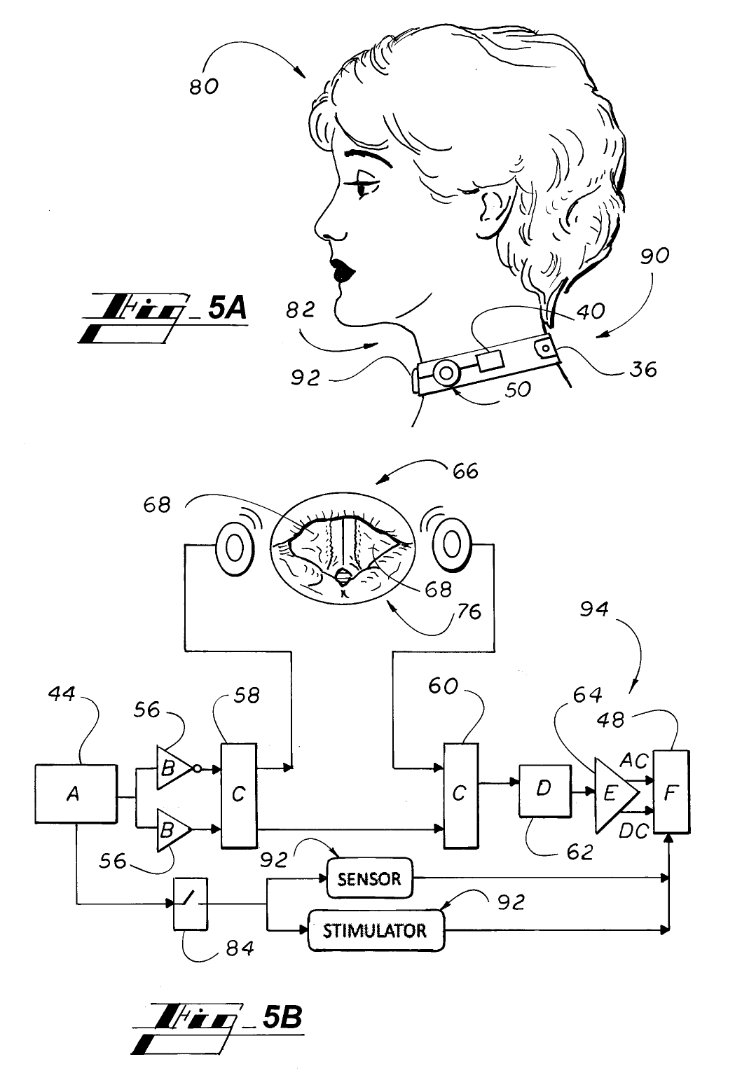

Diagnostic and treatment flow diagram 10 is illustrated in FIG. 2. Diagnostic and treatment flow diagram 10 illustrates earlier diagnosis and treatment of vocal cord dysfunction. In step 12 subject experiences symptoms shared by asthma and VCD, such as airflow obstruction which prevents normal breathing, resulting in wheezing, shortness of breath, and in extreme cases, fainting. In steps 14, 16, and 18 patient 80 (FIG. 5A) (sometimes referred to as subject) is evaluated by medical professionals, such as a primary care physician and/or pulmonary specialist, for, among other things, asthma and VCD at the same time. Under this illustrative embodiment of a diagnosis process, VCD evaluation occurs at nearly the same time or simultaneously with asthma assessment, as illustrated in step 18. With proper VCD diagnosis as illustrated in step 20, patients with VCD would not suffer through a four-year average period of misdiagnosis and cycle through two—three asthma medications and scores of medical visits. In steps 22, 24, patient 80 (FIG. 5A) with VCD diagnosis is treated through behavioral and medical management as well as provided proper follow up visits.

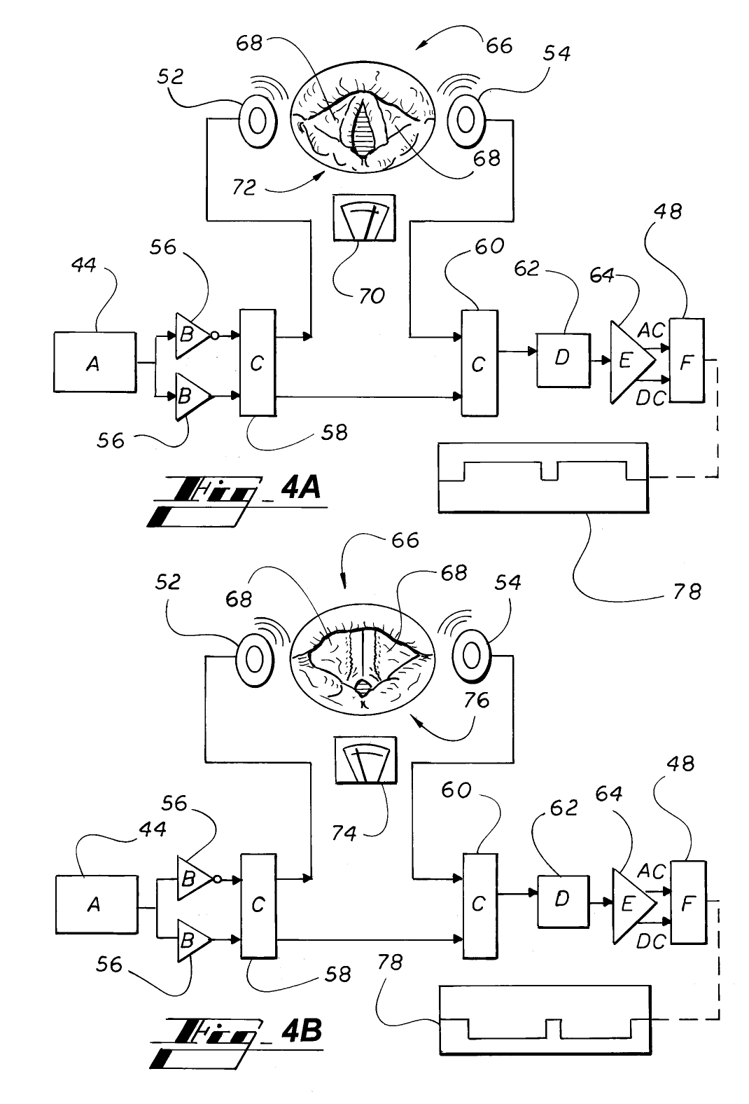

In order to facilitate proper diagnosis without some of the limitations of the current diagnostic options, medical professional may provide patient 80 (FIG. 5A) with portable device 30. Medical professional may evaluate patient 80 by asking patient 80 to use device 30 for a period of time in order to monitor patient 80 and to use device 30 to record any patient 80 respiratory attacks. As described in detail below, device 30 transmits electronic signals between at least one pair of electrodes 50. Device 30 measures and records data of the amount of relative impedance (“resistance”) caused by the area of opening between vocal folds 68 of vocal cords 66 of subject 80. As illustrated in FIG. 4A, a relatively high level of impedance 70 is associated to vocal folds 68 in open position 72. As illustrated in FIG. 4B, a relatively low level of impedance 74 is associated to vocal folds 68 in closed position 76. If patient 80 experiences respiratory attack, device 30 measures and records open position 72 or closed position 76 of vocal cords 66 during respiratory attack. Patient 80 can return recorded data 78 to the medical professional. Recorded data 78 can be transmitted from device 30 to external machine (not shown) for analysis. For example, analysis can be used to determine the degree of closure of vocal cords 66 during respiratory attack, which is an indication of VCD. Recorded data 78 can be used for diagnosis and treatment of vocal cord dysfunction. By not forcing patients to suffer respiratory attacks within a clinical environment, test data is more easily collected. Medical professionals do not waste time trying to induce onsite respiratory attacks or require patients to endure uncomfortable tests. Device 30 increases patient 80 comfort.

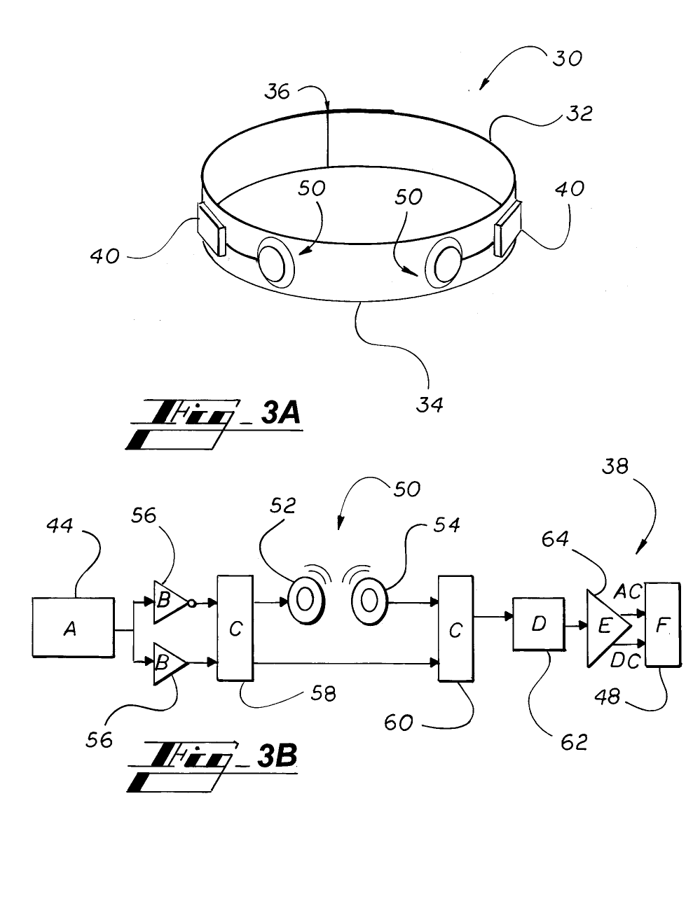

As illustrated in FIG. 3A, device 30 is a noninvasive, portable system based on electroglottograph (EGG) technology. Device 30 uses electrical signals across the larynx (externally on neck 82 (FIG. 5A)) to measure (“gauge”) the relative impedance across vocal cords 66. As previously stated, relative impedance across vocal cords 66 increases when vocal folds 68 are open 72 (FIG. 4A) and relative impedance decreases when vocal folds 68 are closed 76 (FIG. 4B). Relative impedance thereby indicates whether vocal cords 68 are open 72 or closed 76.

With reference to FIG. 3A, device 30 is shown. Device 30 is configured to fit around neck 82 (FIG. 5A) of patient 80 (FIG. 5A). Device 30 is located adjacent to vocal cords 66 of patient 80. Device 30 includes power source 42, signal generator 44, microcontroller 46, at least one pair of electrodes 50, and memory 48.

In this embodiment, device 30 is shown as small, flexible collar 32 which includes strap 34 which includes fastener 36. It is also envisioned that collar 32 and/or strap 34 are fastened to neck 82 (FIG. 5A) of patient 80 (FIG. 5A) in other ways, such as with a zipper, button, elastic material, or the like. As illustrated in FIG. 3A, strap 34 supports circuitry component 40 which includes power source 42, signal generator 44, microcontroller 46, memory 48 and may optionally also include microphone (not shown). Circuitry component 40 is coupled to at least one pair of electrodes 50. Strap 34 is also configured to locate at least one pair of electrodes 50 adjacent to vocal cords 66 of patient 80.

As illustrated in FIG. 3B, circuitry 38 of device 30 is shown in order to illustrate this application. Please note that as part of circuitry 38, at least one pair of electrodes 50 are located on subject's neck 82 with first electrode 52 located on one side of subject's vocal cords 66 and second electrode 54 located on the other side of subject's vocal cords 66. Signal generator 44 is also known as standard clock generator. Signal generator 44 can be designed using a starved-current ring oscillator, a multivibrator, or direct digital synthesis (“DDS”). DDS can be used to generate an analog sinusoidal voltage waveform by generating the signal digitally and then using a digital to analog converter. Signal generator 44 provides an alternating current voltage waveform, such as a sinusoidal waveform. Signal generator 44 may be programmable to any frequency, such as any frequency within the range of approximately 100 kHz to approximately 2 MHz or such as any frequency at least approximately 100 kHz.

The signal is then buffered by a pair of logic circuits 56 in order to generate two phases of the signal that are 180 degrees out of phase with each other. This signal pair is fed into primary coils (not shown) of input transformer 58. First electrode 52 is in parallel with a load capacitor. Secondary transformer 60 is used on second electrode 54 on the other side of neck 82 (FIG. 5A) of patient 80 (FIG. 5A). It is to be noted that as shown in FIG. 3B, one of the secondary terminals of input transformer 58 is shorted to the secondary transformer 60. Neck 82 of patient 80 with tissue and vocal cords 66 forms the capacitor in parallel with secondary transformer 60, allowing for variable impedance in the path depending on the extent of vocal cord closure from open position 72 (FIG. 4A) to closed position 76 (FIG. 4B).

Signal received by secondary transformer 60 is then demodulated using standard amplitude modulated (“AM”) demodulation system 62. The demodulated AM signal is then amplified appropriately as required by the application using standard amplifier 64, which is then filtered to separate the alternating current and the direct current components of the signal. Both components of the signal are stored in memory 48 integrated with circuitry 38 of device 30. Direct current components (sometimes referred to as low-frequency components) of the signal are analyzed for diagnosis of vocal cord dysfunction while EGG typically analyzes the alternating current components (sometimes referred to as high-frequency components) for analysis of speech therapy.

Device 30 may start recording upon trigger 84 (FIG. 5B) or may continually record during a monitoring period. Device 30 continues storing data on memory 48 until interrupted or until memory overflow. A standard connection interface is provided to transfer recorded data on to an external machine, such as a standard computer. The standard computer may use custom built software to transmit, read, and/or analyze recorded data. The software includes a graphical user interface (“GUI”) to upload the data, and a different interface for the physician to analyze the recorded patient data.

Device 30 can be miniaturized. Patients could carry device 30 with them for a period of time, for example two weeks. Device 30 is external and portable, allowing patient 80 (FIG. 5A) to take device 30 with patient 80 during daily life. Device 30 is available to record electronic signals during patient's 80 attack outside of a clinical environment. Data gathered on device 30 is stored on memory, for example a small flash memory drive. Data can then be taken to the doctor's office where a diagnosis can be made. Device 30 eases both the physical and psychological stressors on patient 80 and allows the physician to analyze the data recorded by device 30 as part of diagnosing whether or not patient 80 has vocal cord dysfunction.

As illustrated in FIG. 5A, second embodiment of device 90 is shown. Therapeutic device 90 is similar to diagnostic device 30 in several components. Only the differences will be discussed in detail. Device 90 integrates fully functional miniaturized electrical stimulator 92 to target specific stimulation of the recurrent laryngeal nerve which caused opening of vocal cords 66 or vocal folds 68. There are two envisioned modes of stimulation: a) direct stimulation via implanted cuff electrode and b) non-invasive stimulation using surface electrode. This embodiment incorporates the non-invasive mode of stimulation. Device 90 includes stimulator 92 integrated on collar 32. As illustrated in FIG. 5B, stimulator 92 is integrated into control circuitry 94 along with trigger 84. Device 90 including trigger 84 and stimulator 92 and device 90 illustrates a proposed integrated solution that includes diagnostic and therapeutic intervention schemes on portable collar 32.

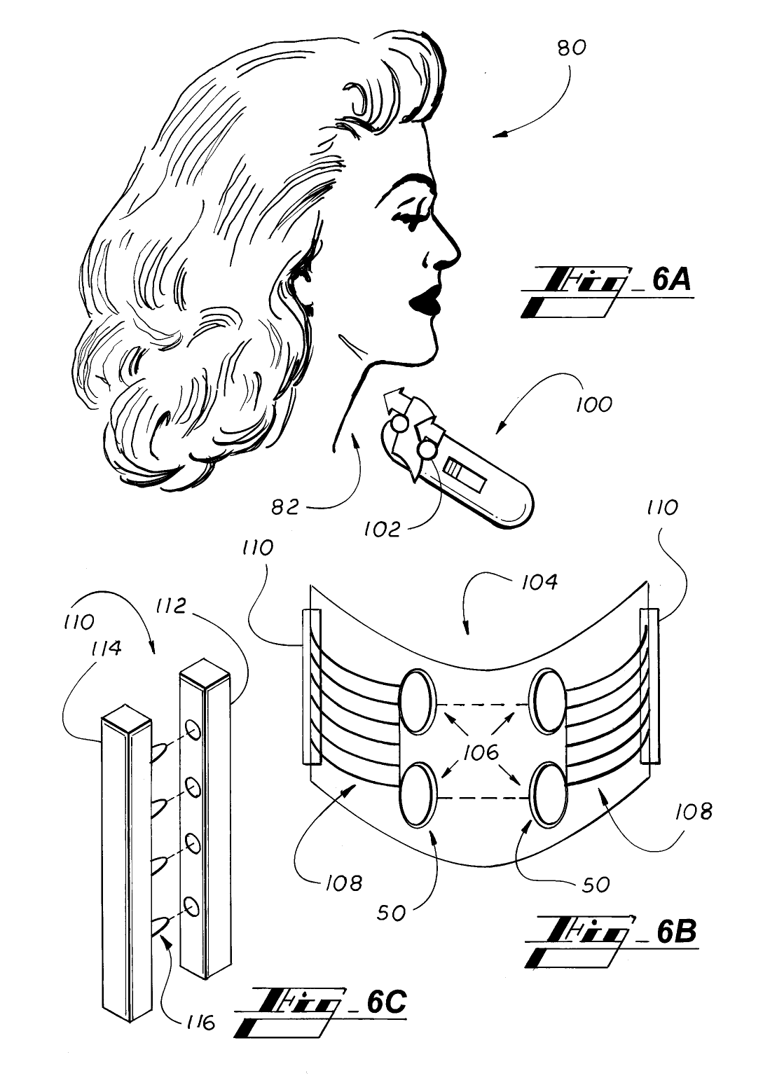

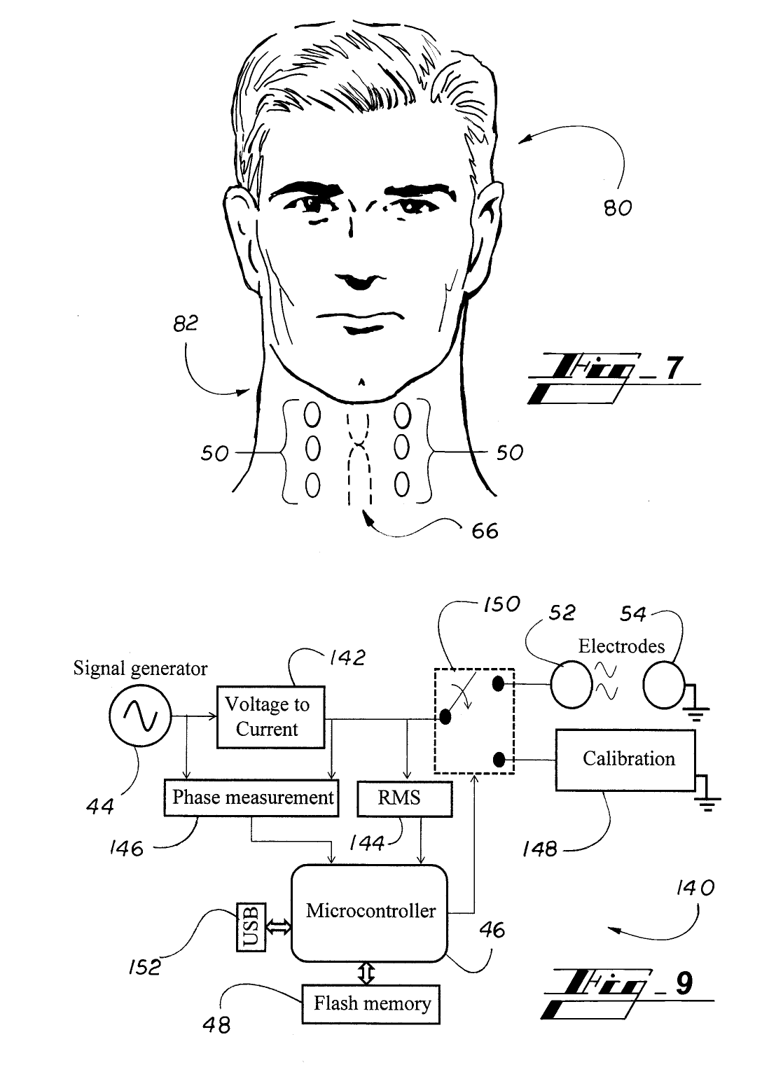

As illustrated in FIG. 6A, third embodiment of device 100 is shown as a hand held unit. Hand held device 100 is similar to collar-based device 30 or therapeutic device 90. The hardware and functionality of these versions is expected to remain identical. Hand held device 100 is envisioned to have removable electrode/recording pads 102 coupled to any form of circuitry 38 or 94. Electrode pads 102 could be pre-coated with electrode gel for application against neck 82 of patient 80. Electrode pads 102 are meant to be disposable after each use. Hand held device 100 may be cordless. This adaption of device 100 is intended for adults that do not wish to strap on collar 32 and also by physicians or caregivers available to monitor attacks by use of device 100.

As illustrated in FIG. 6B, detachable flap 104 is shown for use with any device of the present disclosure, such as collar device 30, hand held device 100 or therapeutic device 90. Detachable flap 104 includes at least one pair of electrodes 50 such as array of electrodes 106. Array of electrodes 106 are configured to be positioned adjacent to vocal cords 66 of patient 80 (FIG. 6A). Detachable flap 104 also includes electrical bus 108 coupling each side of array of electrodes 106 with connector 110. Connector 110 couples detachable flap 104 with the rest of devices 30, 90, 100. As illustrated in FIGS. 6B and 6C, connector 110 includes either female connector 112 or male connector 114. Any one of devices 30, 90, 100 may include the corresponding female connector 112 or male connector 114 in order to connect with detachable flap 104. Female and male connectors 112, 114 illustrate four circuit connection points 116. It is envisioned that female and male connectors 112, 114 can include any number of connection points 116. As illustrated in FIG. 7, it is envisioned that detachable flap 104 can include any plurality of electrodes 50. Similarly, it is also envisioned that any of devices 30, 90, 100 may incorporate any plurality of electrodes 50 as illustrated in FIG. 7. An increased number of paired electrodes 50 could be beneficial to ensure any one of devices 30, 90, 100 is able to measure vocal cords 66.

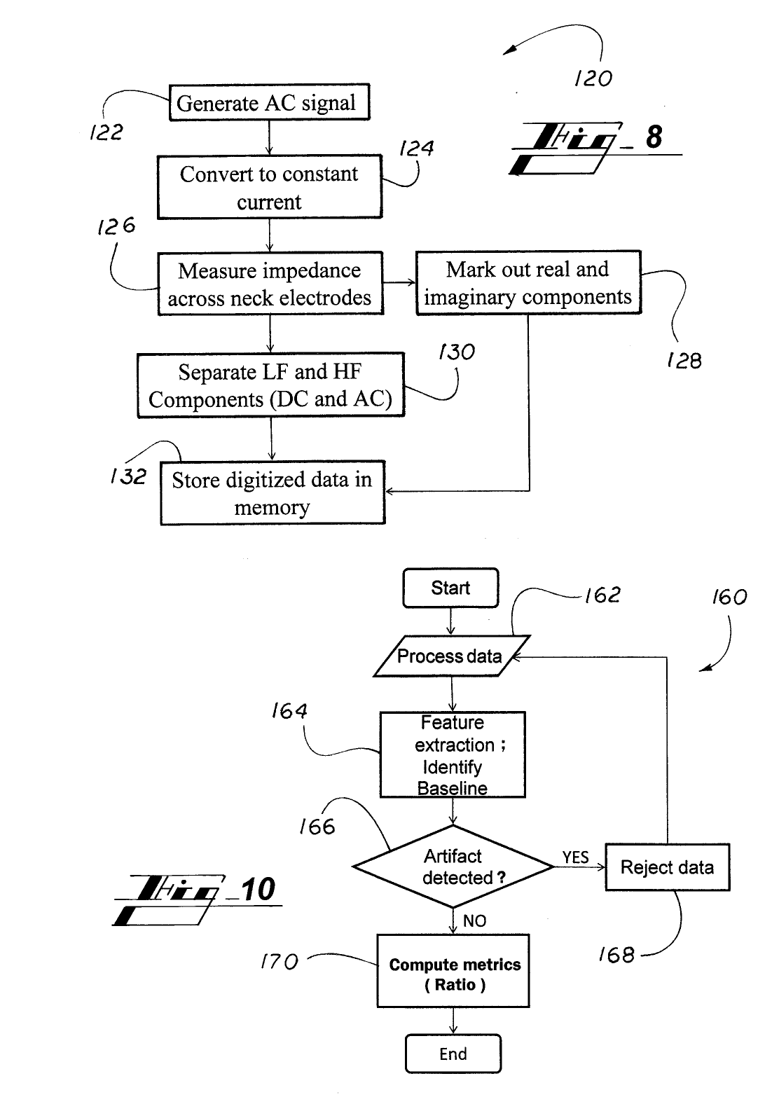

As illustrated in FIG. 8, flow diagram 120 illustrates the steps performed in circuitry 140 (FIG. 9). In step 122, an alternating current signal is generated. In step 124, the alternating current signal is converted into constant current amplitude with an alternating voltage waveform. In step 126, this waveform is transmitted across vocal cords 66 of patient 80 (FIG. 6A) and is measured to determine the magnitude of relative impedance across vocal cords 66. Relative impedance across vocal cords 66 increases when vocal cords 66 are open 72 (FIG. 4A) and decreases when vocal cords 66 are closed 76 (FIG. 4B). In step 128, real and imaginary components are marked out. In step 130, low frequency (direct current) and high frequency (alternating current) components are separated. In step 132, the separate components are stored as digitized data in memory. The digitized data can be used by a medical professional to diagnose patient 80 with vocal cord dysfunction.

As illustrated in FIG. 9, circuitry 140 is shown in order to illustrate an embodiment of flow diagram 120 (FIG. 8) and to illustrate application with any of devices 30, 90, 100. Please note that as part of circuitry 140, at least one pair of electrodes 50 are located on subject's neck 82 with first electrode 52 located on one side of subject's vocal cords 66 and second electrode 54 located on the other side of subject's vocal cords 66. Signal generator 44 provides an alternating current voltage waveform, such as a sinusoidal waveform. The alternating current voltage waveform is converted to constant current amplitude using voltage-to-current converter 142, such as voltage controller current source 142. The constant current amplitude and alternating voltage waveform is then transmitted across vocal cords 66 of patient 80 (FIG. 6A).

The constant current amplitude and alternating voltage waveform is measured by root-mean-square detector 144 to gauge the relative impedance of vocal cords 66. The root mean square value of the voltage measured across electrodes 52, 54 is computed to evaluate the magnitude of the relative impedance. Relative impedance across vocal cords 66 increases when vocal folds 68 are open 72 (FIG. 4A) and decreases when vocal folds 68 are closed 76 (FIG. 4B). Relative impedance thereby indicates the area of opening between vocal folds 68 of vocal cords 66, whether vocal folds 68 of vocal cords 66 are opened 72 or closed 76. The phase (imaginary component) measuring system 146 of the measured relative impedance would facilitate in assessing the resistive and capacitive components to the magnitude of relative impedance change.

Diagnostic data from electrode signals is then digitized and is stored in memory 48 controlled by microcontroller 46. Calibration module 148 is attached to microcontroller 46 to allow for periodic impedance calibration. The calibration phase is enabled by microcontroller 46 using switch 150 that routes the current in a parallel path away from electrodes 52, 54. Microcontroller 46 is coupled to an external machine, such as a standard computer, using interface 152, such as a USB or other compatible interfaces. Interface 152 allows for data stored in memory 48 to be downloaded and analyzed by a medical professional in order to diagnose patient with vocal cord dysfunction.

In FIG. 10, flow diagram 160 illustrates software process steps for use with stored data according to any embodiment of the present disclosure. In step 162, software processes diagnostic data. In step 164, software extracts and identifies a baseline. In step 166, software determines whether or not an artifact is detected. If step 166 is yes, then in step 168, software rejects data and returns to step 162. If step 166 is no, then in step 170, software calculates metrics and reports results to a user, such as medical professional for diagnosis of vocal cord dysfunction.

While this disclosure has been described as having an exemplary design, the present disclosure may be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains.

What is claimed is:

1. A device supported by a subject or by an investigator, the device using electronic signals to provide diagnostic data indicating an area of opening between vocal folds of vocal cords of subject, whether vocal folds of vocal cords are opened or closed as an indication of vocal cord dysfunction, the device comprising:
a power source,
a signal generator powered by the power source, the signal generator configured to generate electronic signals,
a microcontroller configured to transmit, direct, or control the passage of electronic signals,
at least one pair of electrodes, the at least one pair of electrodes configured to be disposed adjacent to the vocal cords of the subject, the at least one pair of vocal cords including a first electrode configured to be disposed to one side of the vocal cords and a second electrode configured to be disposed to the other side of the vocal cords, wherein the transmission of electronic signals between the at least one pair of electrodes and through the vocal cords of the subject provides diagnostic data indicating an open position or a closed position of the subject's vocal cords, and
memory configured to record diagnostic data.

2. The device of claim 1 further comprising a strap configured to fit around the neck of the subject, the strap configured to fit adjacent to the vocal cords of the subject, the strap including a fastener, the strap configured to support the power source, the signal generator, the microcontroller, the at least one pair of electrodes and memory.

3. The device of claim 2, wherein the strap is configured to locate the at least one pair of electrodes adjacent to the vocal cords of the subject.

4. The device of claim 1, wherein the device is a hand held unit supported by the subject, the investigator, or a caregiver.

5. The device of claim 1 further comprising a user trigger, a sensor, and a stimulator coupled to the power source, wherein the stimulator is configured to stimulate of the recurrent laryngeal nerve of the patient causing opening of the vocal cords of the patient.

6. The device of claim 1, wherein the at least one pair of electrodes is supported by a detachable flap.

7. The device of claim 6, wherein the detachable flap includes connectors to connect the detachable flap with the signal generator and the microcontroller, the connectors configured to transmit electronic signals to and from the at least one pair of electrodes.

8. The device of claim 1, further comprising a microphone coupled to the memory, the microphone configured to record sounds from the subject in conjunction with the diagnostic data.

9. A method of diagnosing vocal cord dysfunction by recording the position of the vocal cords of a subject during a monitoring period, the method comprises the steps of:
evaluating a subject for asthma or vocal cord dysfunction,
providing the subject with a recording device which measures the area of opening between vocal folds of the vocal cords, the device including at least one pair of electrodes configured to be disposed on opposite sides of the vocal cords of the subject,
monitoring the subject for a period of time,
transmitting electronic signals between the at least one pair of electrodes,
recording data indicating an open position or a closed position of the subject's vocal cords during the monitoring period of time, and
analyzing data to diagnose vocal cord dysfunction.

10. The method of claim 9 further comprising the step of treating the subject with vocal cord dysfunction by stimulating a nerve which causes opening of the vocal cords.

11. The method of claim 9 further comprising the step of recording the position of the vocal cords of the subject during a period of airflow obstruction during the monitoring period of time.

12. The method of claim 9 further comprising the step of transmitting data to an external machine.

13. The method of claim 9 wherein the device includes a collar to support the device on the subject.

14. The method of claim 13 wherein the step of providing the subject with the recording device further comprises the step of fitting the subject with the recording device, wherein the recording device is configured to fit around to the neck of the subject.

15. The method of claim 9 wherein the device is a hand held unit supported by the subject, an investigator, or a caregiver.

16. The method of claim 15 wherein the step of providing the subject with the recording device further comprises the step of fitting the subject with at least a portion of the recording device, wherein the portion of the recording device is configured to be disposed adjacent to the vocal cords of the subject.

17. A device using electronic signals to provide diagnostic data indicating a subject's closed or open vocal cords during vocal cord dysfunction, the device comprising:
a signal generator configured to generate a sinusoidal AC voltage waveform,
a voltage-to-current converter configured to generate a constant current amplitude and alternating voltage,
a switch configured to couple the constant current amplitude and alternating voltage to an electrode,
a pair of electrodes configured to transmit the constant current amplitude and alternating voltage across the vocal cords of the subject,
a root-mean-square detector to measure the relative impedance across the vocal cords,
a microcontroller configured to transmit, direct or digitize the measured relative impedance, and
memory configured to store electronic signals.

18. The device of claim 17 further comprising a phase measurement component for comparison against the measured relative impedance.

19. The device of claim 17 further comprising a calibration module coupled to the microcontroller by the switch.

20. The device of claim 17 further comprising an interface which allows for stored electronic signals to be downloaded.
